(19) United States
(12) Patent Application Publication
(10) Pub. No.: US 2026/0130447 A1
(43) Pub. Date: May 14, 2026

(54) System for Reconfigurable Donning of a Hand Orthotic Device

(71) Applicant: Myomo, Inc., Boston, MA (US)

(72) Inventors: Jake A. Schwartz, Boston, MA (US); David R. Coleman, Madison Lake, MN (US); Andrew Harlan, Somerville, MA (US); Dan Echeverria, Cambridge, MA (US)

(21) Appl. No.: 18/941,131

(22) Filed: Nov. 8, 2024

(51) Int. Cl. A41D 13/08; A61F 5/01

(52) U.S. Cl. CPC A41D 13/087; A61F 5/013

(57) ABSTRACT

A hand module of an orthotic device assists a user with an impaired ability to grasp and pick up objects. The hand module comfortably secures a user's hand in a hand shell and is configurable by a clinical professional at the time of delivery for one of two different donning methods, a top-down method and a slide-through method. In the top-down (or dorsal) method, a user's palm is positioned on top of the hand shell and strapped in from above. In the slide-through (or thenar) method the user's hand is pulled through a pre-formed upper loop and strapped around the user's thumb from below. The hand module is configured with a grasp actuator that can move between an open latched position and a closed unlatched position by pushing a lever or by pulling on the grasp actuator with force exceeding a threshold value.

### TECHNICAL FIELD

The present disclosure relates to orthotic devices for assisting the motions of users with limb impairments. In particular, the disclosure relates to methods of donning such devices.

### BACKGROUND ART

An orthotic device with a hand module may provide assistance to and facilitate the rehabilitation of a user with an impaired ability to grasp and pick up objects. Such a user may have specific challenges that make unassisted donning of the orthotic device slow and difficult.

### SUMMARY OF THE EMBODIMENTS

Examples of embodiments are provided below. These example are non-exhaustive, as other embodiments may be implemented.

In accordance with an embodiment of the invention, an orthotic device is disclosed, the orthotic device having a hand module configured for strapping a hand of a user, and for effectuating relative movement of a thumb of the hand and a set of fingers of the hand, the hand module including a hand shell, configured to receive a palm of the hand, a finger shell, coupled to the hand shell, and configured to receive the set of fingers, a thumb shell, coupled to the hand shell, and configured to receive the thumb, and a hand strapping system. In this embodiment, the finger shell and the thumb shell are configured to support relative motion of the thumb and the set of fingers, and a grasp actuator is coupled to the finger shell and the thumb shell, and configured to cause relative motion of the set of fingers and the thumb. The hand strapping system functions to secure a dorsal side of the user's hand and a base of the user's thumb, the hand strapping system including mounted hardware to support (i) a dorsal strapping arrangement for top-down donning and (ii) a thenar strapping arrangement for slide-through donning.

The dorsal strapping arrangement includes a dorsal ring, removably attached on a radial region of the hand shell, a thenar loop, configured to engage the base of the user's thumb with the user's thumb received in the thumb shell, and a dorsal strap configured to engage against the dorsal side of the hand and to be looped through the dorsal ring, and thence back to itself. The dorsal strap in this dorsal strapping arrangement can be removably attached, so as to secure the hand between the dorsal strap on the dorsal side of the hand and the hand shell on the palm of the hand.

The thenar strapping arrangement includes a volar ring, removably attached on the radial region of the hand shell, a dorsal loop, configured to engage against the dorsal side of the user's hand with the palm received on the hand shell, and a thenar strap configured to wrap around the base of the thumb and to be looped through the volar ring, and thence back to itself. The thenar strap can be removably attached, so as to secure the thumb in the thumb shell.

In an embodiment, the hand shell has an ulnar region, configured such that with the hand of the user secured in the hand module, the ulnar region is disposed adjacent to a little finger of the hand, the ulnar region having an ulnar slot configured to allow passage of a strap inserted therein, such that in the dorsal strap arrangement, the dorsal strap is configured to pass through the ulnar slot, in a dorsal to volar direction, and to be affixed to the radial region of the hand shell, thereby forming the thenar loop. In the thenar strap arrangement, the thenar strap is configured to pass through the ulnar slot, in a volar to dorsal direction, and to be affixed to the radial region of the hand shell, thereby forming the dorsal loop.

According to some embodiments, the ulnar slot has a perimeter with features configured to provide a frictional hold on the dorsal strap with the dorsal strapping arrangement and on the thenar strap with the thenar strapping arrangement. According to some such embodiments, the features configured to provide the frictional hold are teeth.

According to some embodiments, a wrist shell is configured to secure a wrist of the user with the hand secured in the hand shell, and a hand to wrist coupler connecting the wrist shell to the hand shell by a palm swivel joint on the hand shell, the palm swivel joint configured to allow swiveling of the hand about a dorsal to volar axis centered at the palm swivel joint.

In an embodiment, the orthotic device has a dorsal mounting feature, the dorsal ring being attached to the dorsal mounting feature in the dorsal strapping arrangement. In an embodiment, the orthotic device has a volar mounting feature, the volar ring being attached to the volar mounting feature in the thenar strapping arrangement.

According to some embodiments, the thenar loop is attached to the volar mounting feature for the dorsal strapping arrangement. According to some embodiments, the dorsal loop is attached to the dorsal mounting feature for the thenar strapping arrangement.

According to some embodiments, the dorsal mounting feature has a dorsal ring tightening mechanism which allows a frictional force on the dorsal ring to be adjusted, allowing the dorsal ring to be held in an open position as the dorsal strap is passed through it.

According to some embodiments, the dorsal strap and the thenar strap are each configured with a hook-and-loop mechanism to allow the dorsal strap to removably attach to itself, and the thenar strap to removably attach to itself.

In an embodiment, an orthotic device having a hand module is configured for strapping a hand of a user, and for effectuating relative movement of a thumb of the hand and a set of fingers of the hand, the hand module including a hand shell configured to receive a palm of the hand, a finger shell, coupled to the hand shell, and configured to receive the set of fingers, a thumb shell, coupled to the hand shell, and configured to receive the thumb, a hand strapping system, configured to secure the hand in the hand module, and a grasp actuator. For this embodiment, the finger shell and the thumb shell are configured to support relative motion of the thumb and the set of fingers. For this embodiment, the grasp actuator has a pivotal attachment to a radial region of the hand shell, the pivotal attachment being configured to allow the grasp actuator to pivot about a hinge from a closed configuration to an open configuration, and to hold the grasp actuator in place in the closed configuration and in the open configuration. For this embodiment, the open configuration allows the hand to be moved into place with the palm received by the hand shell, the set of fingers received by the finger shell, and the thumb received by the thumb shell. The closed configuration allows operation of the grasp actuator to cause relative motion of the fingers and the thumb. For this embodiment, the orthotic device further includes an actuator lever mechanically coupled to the grasp actuator, and a self-opening mechanism configured to move the grasp actuator from the closed configuration to the open configuration in response to a trigger action by the user. According to some embodiments, the trigger action is selected from the group consisting of pressing the actuator lever and applying a force in an opening direction to the grasp actuator. According to some embodiments, the applied force must be at least 13 Newtons.

According to some embodiments, the self-opening mechanism is equipped with torsional springs about the hinge, the torsional springs being taut in the closed configuration of the grasp actuator, for which application of the action releases tension on the torsional springs, causing the grasp actuator to transition from the closed configuration to the open configuration. According to some embodiments, the self-opening mechanism is effectuated by a cam latch.

In an embodiment, a method of securing a hand of a user in the hand module of an orthotic device is disclosed, the method including:
(1) pursuant to choosing the dorsal strapping arrangement:
(2) placing the palm of the hand on a palm region of the hand shell;
(3) positioning the thumb through the thenar loop into the thumb shell;
(4) passing the dorsal strap over a dorsal region of the hand;
(5) inserting the dorsal strap in a radial direction through the dorsal ring;
(6) passing the dorsal strap through the dorsal ring;
(7) folding the dorsal strap back towards the little finger;
(8) pulling the dorsal strap towards the little finger; and
(9) attaching the dorsal strap to itself, thereby securing the hand in the hand module.

In an embodiment, a method of securing a hand of a user in a hand module of an orthotic device is disclosed, the method including:
(1) pursuant to choosing the thenar strapping arrangement:
(2) sliding the hand of the user between the dorsal loop and a palm region of the hand shell;
(3) positioning the thumb of the user in the thumb shell;
(4) passing the thenar strap around the base of the thumb;
(5) inserting the thenar strap in a radial direction through the volar ring;
(6) passing the thenar strap through the volar ring;
(7) folding the thenar strap back towards the little finger;
(8) pulling the thenar strap towards the little finger; and
(9) attaching the thenar strap to itself, thereby securing the hand in the hand module.

### BRIEF DESCRIPTION OF THE DRAWINGS

The foregoing features of embodiments will be more readily understood by reference to the following detailed description, taken with reference to the accompanying drawings.

FIG. 1 shows directions referenced to a position of a hand when secured in a hand module of an orthotic device.

FIG. 2 provides a dorsal to volar view of a hand module as disclosed herein, with an associated hand strapping system removed, and a hand actuator in an open position.

FIG. 3 provides a view of the hand module of FIG. 2, in a radial direction from an ulnar side of the hand module, with the hand actuator in a closed position.

FIG. 4 provides another view of the hand module of FIG. 2, in an ulnar direction from a radial side of the hand module.

FIG. 5 provides a view of the hand module of FIG. 2, shown in a dorsal direction from a volar side of the module.

FIG. 6 provides another view of the hand module of FIG. 2, with attached dorsal and volar rings, with the actuator in the open position.

FIG. 7 provides a view of the hand module of FIG. 2, from an actuator side of the module, with the actuator in the open position.

FIG. 8 shows a top-down view of a hand module in a dorsal configuration comprising a hand shell configured with straps and a dorsal ring to accept a hand onto the hand shell.

FIG. 9 provides a view of the hand module of FIG. 8, in a proximal to distal direction.

FIG. 10 shows a view of the hand module of FIG. 8, with a dorsal strap pulled through the dorsal ring, and folded back and attached to itself.

FIG. 11 provides a view of the hand module of FIG. 8, showing how a user's hand moves into position on the hand shell and the thumb moves into the thumb shell and dorsal loop.

FIG. 12 provides a view of the hand module in a thenar configuration comprising a hand shell configured with straps and a volar ring to accept a hand onto the hand shell.

FIG. 13 provides a view of the hand module of FIG. 12, with a thenar strap pulled through the volar ring, and folded back and attached to itself.

FIG. 14 provides a view of the hand module of FIG. 12, showing how a user's hand slides into position between the hand shell and the dorsal loop in the thenar configuration.

FIG. 15 shows a close-up view of an ulnar section of a palm region of the hand shell, highlighting an ulnar slot and protrusions projecting from a perimeter of the ulnar slot.

FIG. 16 shows a close-up view of the hand shell, highlighting a dorsal mounting feature on a radial region of the hand shell.

FIG. 17 shows a close-up view of the palm region of the hand shell, highlighting a volar mounting feature.

FIGS. 18A, 18B, 18C, 18D demonstrate donning the hand module in a top-down method for a dorsal configuration of the hand module with a grasp actuator unlatched and rotated to a radial position and a hand placed palm-down, with a dorsal strap folded over a dorsal part of the hand, with the grasp actuator rotated to a dorsal position and latched closed, and with the hand rotated into place in a wrist shell, respectively.

FIGS. 19A, 19B, 19C demonstrate donning the hand module by sliding a user's hand through a preformed dorsal loop in the thenar configuration of the hand module with the grasp actuator unlatched and rotated wo an open position and a hand slid onto a palm region of a hand shell, with the grasp actuator rotated to the dorsal position and latched in place, and with a thenar strap folded over a base of a thumb and back on itself and removably attached to itself, respectively.

FIG. 20 shows a closed configuration of a grasp actuator on the hand shell, with a user's free thumb positioned on an actuator lever.

FIG. 21 shows the open configuration of the grasp actuator on the hand shell, after opening caused by pressing the user's free thumb on the actuator lever.

FIG. 22 shows a closed configuration of the grasp actuator on the hand shell, with a user's free hand positioned on the grasp actuator.

FIG. 23 shows the open configuration of the grasp actuator on the hand shell, after opening caused by pulling on the grasp actuator with the user's free hand.

FIG. 24 shows a locked configuration of the grasp actuator equipped with a cam latch, a hinge, and torsional springs.

FIG. 25 shows the grasp actuator of FIG. 24, after opening by pushing the actuator lever or by pulling on the grasp actuator.

FIG. 26 shows a cross-section of a cam latch shown in FIG. 24.

### DETAILED DESCRIPTION OF SPECIFIC EMBODIMENTS

Definitions. As used in this description and the accompanying claims, the following terms shall have the meanings indicated, unless the context otherwise requires:

A “volar” section of a hand is the palm of the hand.

A “dorsal” section of the hand is the side of the hand opposite the palm of the hand.

A “volar” direction is the direction from the dorsal section of the hand to the palm of the hand.

A “dorsal” direction is the direction from the palm of the hand to the dorsal section of the hand.

A “thenar” section of the hand is the rounded fleshy part of the hand at the base of the thumb.

An “ulnar” side of the hand is the side on which the little finger (pinky) is disposed.

A “radial” side of the hand is the thumb side of the hand.

A “thenar” direction is the direction from the ulnar side of the hand to the thenar side of the hand.

An “ulnar” direction is the direction from the radial side of the hand to the ulnar side of the hand.

A “radial” direction is the direction from the ulnar side of the hand to the radial side of the hand.

A “distal” direction is from the palm to the tip of a finger.

A “proximal” direction is from the tip of a finger to the palm.

A “set” has at least one element.

“Hook and loop” fastening (e.g. with Velcro® hook-and-loop fasteners) is a means of attaching two surfaces to each other which allows for the two surfaces to be repeatedly fastened and unfastened.

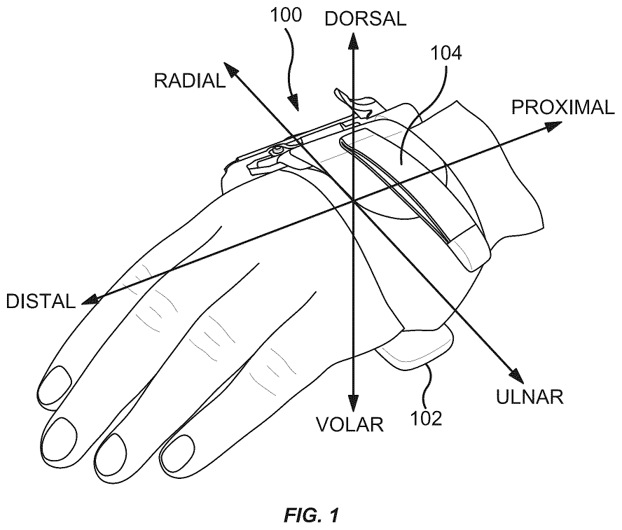

FIG. 1 shows a user's hand strapped into a hand module 100 of an orthotic device, the hand module including a hand shell 102 and a hand strapping system 104. Directional definitions related to a hand are illustrated in FIG. 1.

Directions related to the hand module 100 are defined by the directions of the hand when properly positioned in the hand module 100 as shown in FIG. 1.

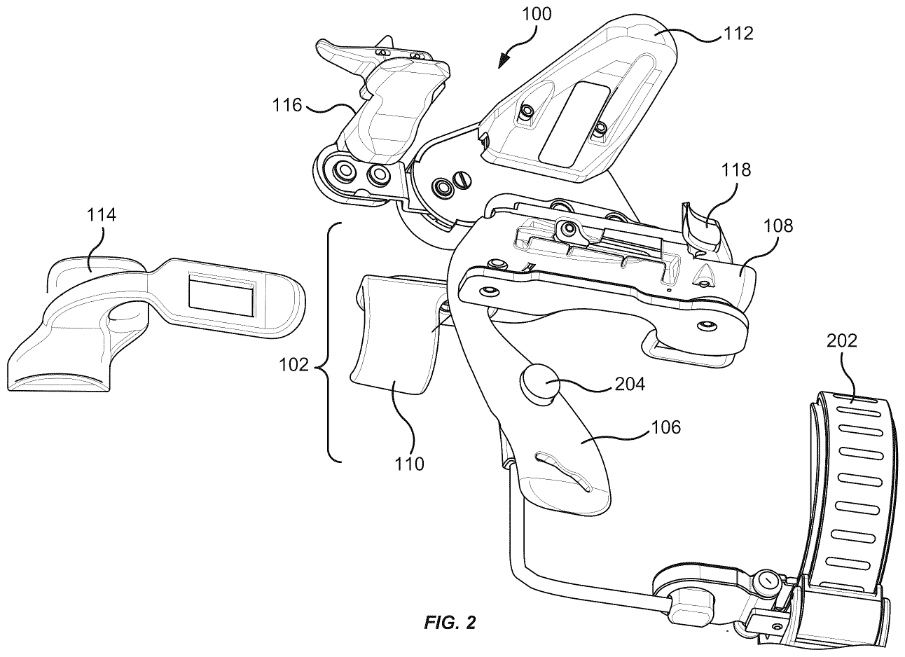

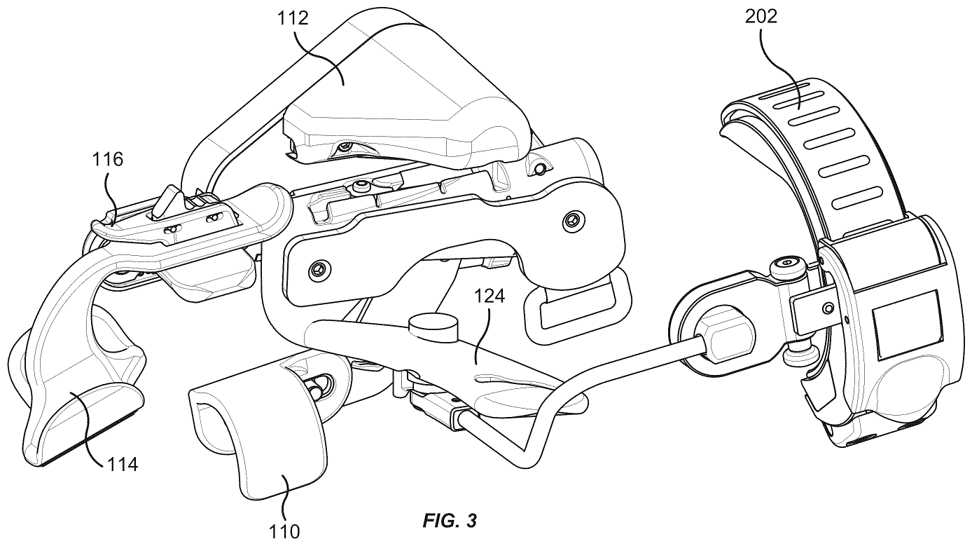

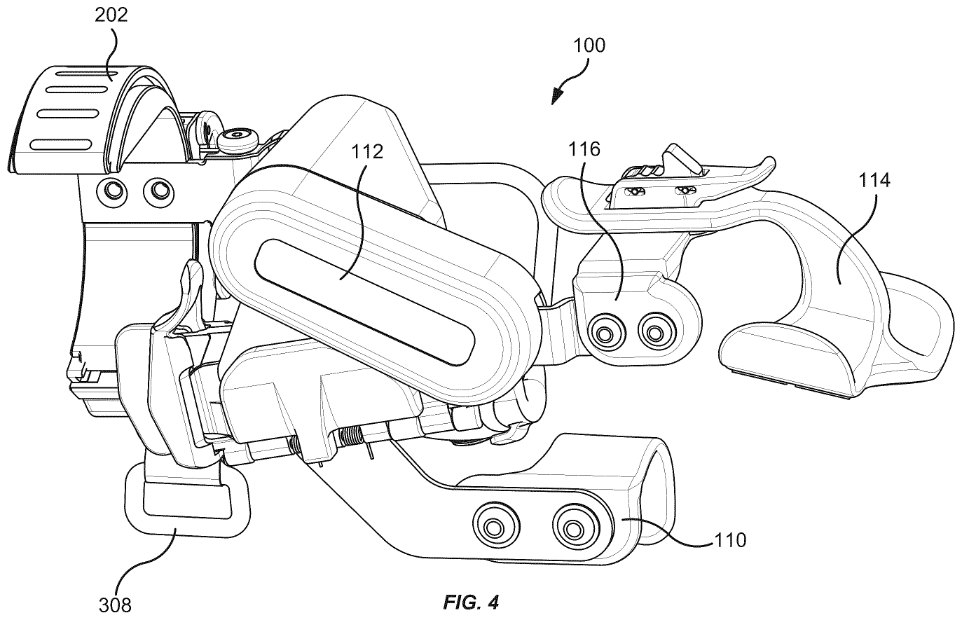

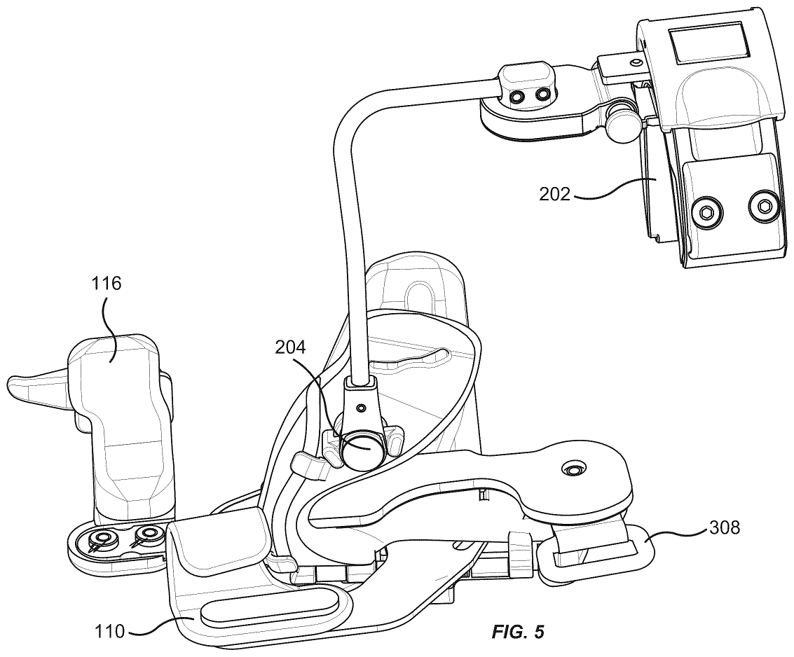

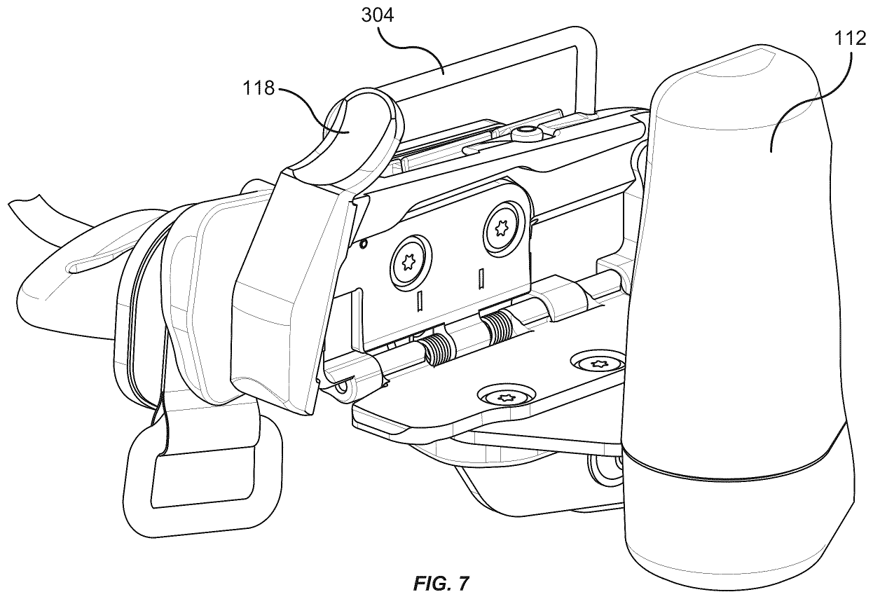

As embodied in FIGS. 2-7 the hand module 100 includes a hand shell 102 having a palm region 106 configured to support the palm of a user's hand, and a radial region 108 configured to be situated between the index finger and the thumb of the hand, abutting the index finger. The hand shell 102 is pivotally attached to a wrist shell 202 of the orthotic device by a rotatable palm swivel joint 204 at or near a center of the palm region 106 of the hand shell 102, the palm swivel joint 204 allowing swiveling of the hand and thus of an arm attached to the hand about a dorsal to volar axis centered at the palm swivel joint 204. The hand module 100 further includes a thumb shell 110 configured to receive and engage the user's thumb, and a grasp actuator 112, attached to the radial region 108 of the hand shell 102. The grasp actuator 112 includes a distal region to which a finger shell mounting feature 116 is attached and a proximal region having an actuator lever 118. A finger shell 114 is removably attachable to the finger shell mounting feature 116. As discussed in more detail below, pressing the actuator lever 118 unlatches the grasp actuator 112 and causes the grasp actuator 112 to rotate from a latched dorsal position (as shown in FIGS. 3, 4, and 5) to a radial position with respect to the hand shell 102 (as shown in FIGS. 2, 6, and 7) so as to allow access to the palm region 106 of the hand shell 102 by the user's hand.

User-specific 3D printing may be used to manufacture the hand shell 102, the thumb shell 110, and the finger shell 114. In this manner, these components can be tailored to fit a range of hand sizes and shapes.

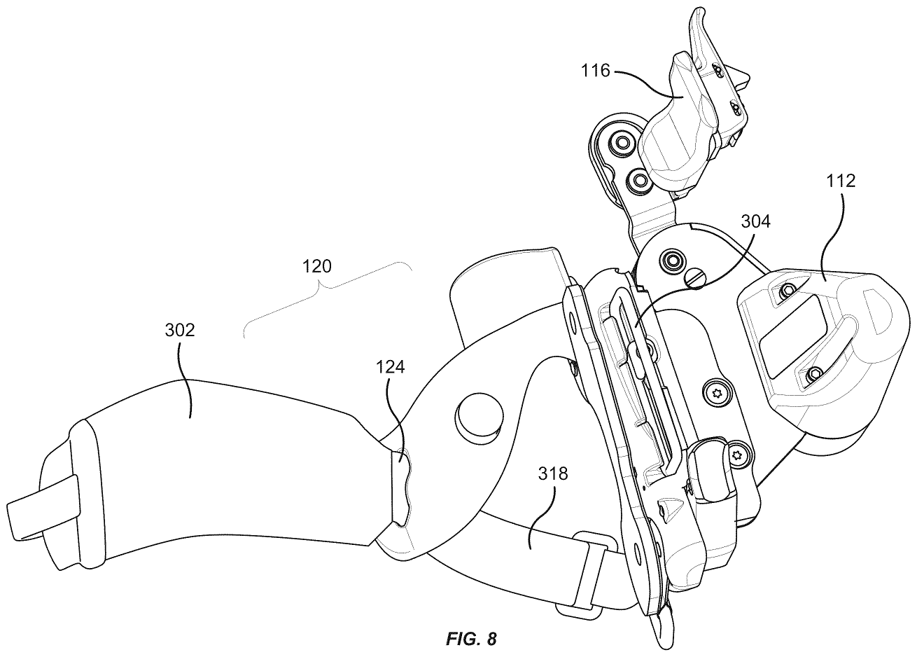

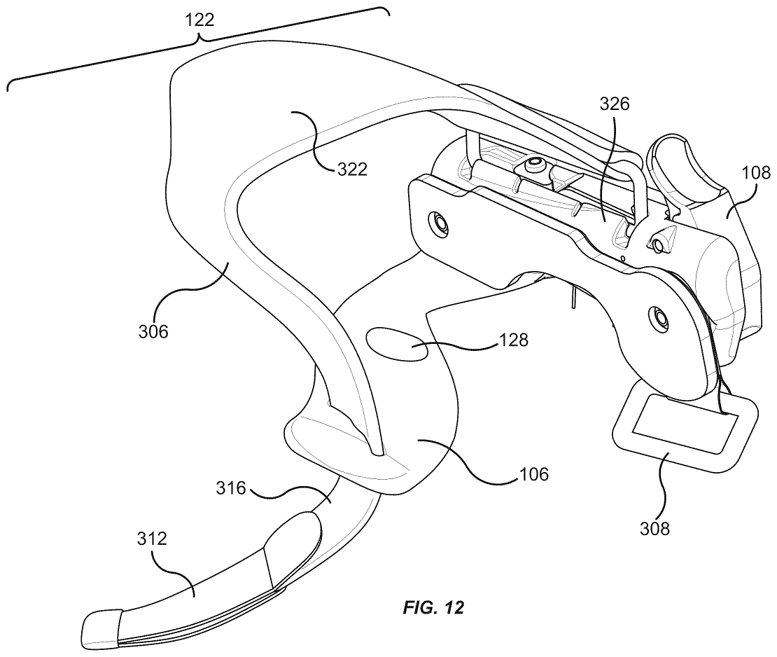

As embodied in FIGS. 8-14, the hand module 100 is designed to comfortably secure a user's hand to the orthotic device by a hand strapping system 104. The hand strapping system may be configurable, e.g. at the time of delivery by a clinical professional, to help support a user's specific donning challenges. For example, the hand strapping system 104 includes a dorsal strapping arrangement 120, embodied in FIGS. 8-11, and a thenar strapping arrangement 122, embodied in FIGS. 12-14. The dorsal strapping arrangement 120 is configured with a dorsal strap 302, a dorsal ring 304, and a thenar loop 318 to facilitate a top-down donning procedure as shown in FIGS. 8-11 and 18. For a slide-through donning procedure as shown in FIG. 12-14, the thenar strapping arrangement 122 is configured with a thenar strap 306, a volar ring 308, and a dorsal loop 322.

In the embodiment of FIGS. 8-14, the dorsal strap 302 and the thenar strap 306 are each custom length C-fold soft-good straps made of a loop material for hook and loop fastening, having a dorsal alligator tab 310 and a thenar alligator tab 312, respectively, with hooks for hook and loop fastening, allowing the dorsal alligator tab 310 to fasten to dorsal strap loop material 314 and the thenar alligator tab 312 to fasten to thenar strap loop material 316.

For the embodiment of FIGS. 8-14, with further reference to FIG. 15, the palm region 106 of the hand shell 102 has an ulnar slot 124 positioned in an ulnar region of the hand shell 102, configured to allow passage of the dorsal strap 302 between the dorsal and volar sides of the hand shell 102 in the dorsal strapping arrangement 120, and to allow passage of the thenar strap 306 between the volar and dorsal sides of the hand shell 102 in the thenar strapping arrangement 122. As shown the expanded view of FIG. 15, the perimeter of the ulnar slot 124 may have at least one protrusion 126 providing a frictional hold on a strap passing through the ulnar slot 124. Each of the at least one protrusion 126 may comprise a set of teeth to provide a frictional hold on the strap. This frictional hold functions as a “checkpoint” which enables a user to maintain orthotic device donning progress by maintaining the position of the strap after it is pulled to tighten.

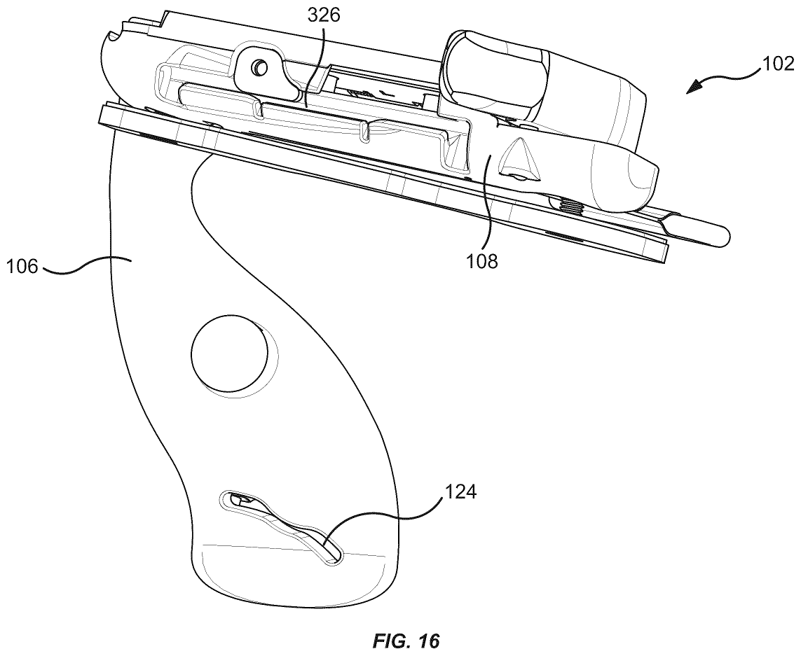

As shown in FIG. 16, for the dorsal strapping arrangement 120 of FIGS. 8-14, the dorsal side of the radial region 108 of the hand shell 102 may have a dorsal mounting feature 326 configured to mount the dorsal ring 304 in the dorsal strapping arrangement 120. This same dorsal mounting feature 326 may also facilitate attachment of a dorsal segment of the thenar strap 306, which forms the dorsal loop 322 in the thenar strapping arrangement 122.

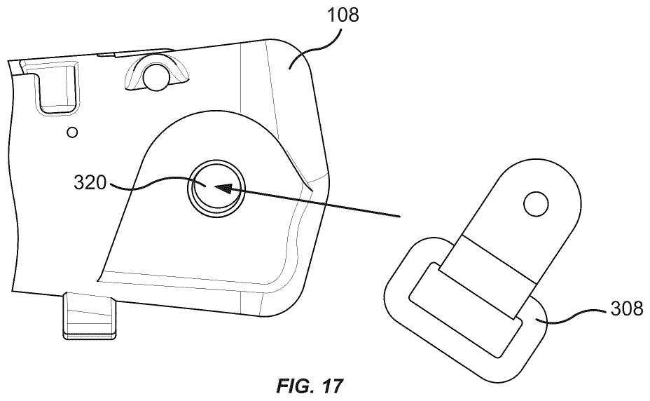

As shown in FIG. 17, for the thenar strapping arrangement of FIGS. 12-14, the volar side of the radial region 108 of the hand shell 102 may have a volar mounting feature 320 configured to mount the volar ring 308 in the thenar strapping arrangement 122. This same volar mounting feature 320 may facilitate attachment of a volar segment of the dorsal strap 302, which forms the thenar loop 318 in the dorsal strapping arrangement 120.

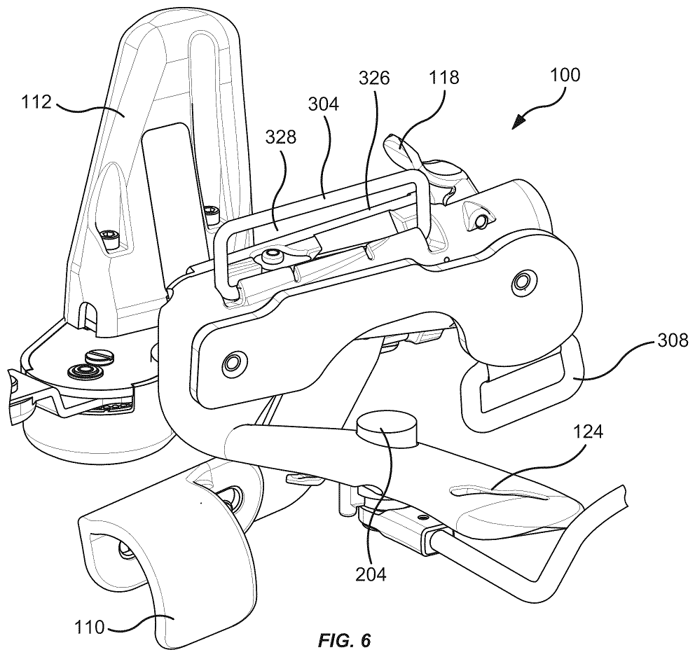

Referring back to FIG. 6, in the dorsal strapping arrangement 120 a tightening mechanism, provided by a dorsal ring mounting screw 328, allows a frictional force on the dorsal ring 304 to be adjusted, to help hold the dorsal ring 304 in an open position as the dorsal strap is threaded through it.

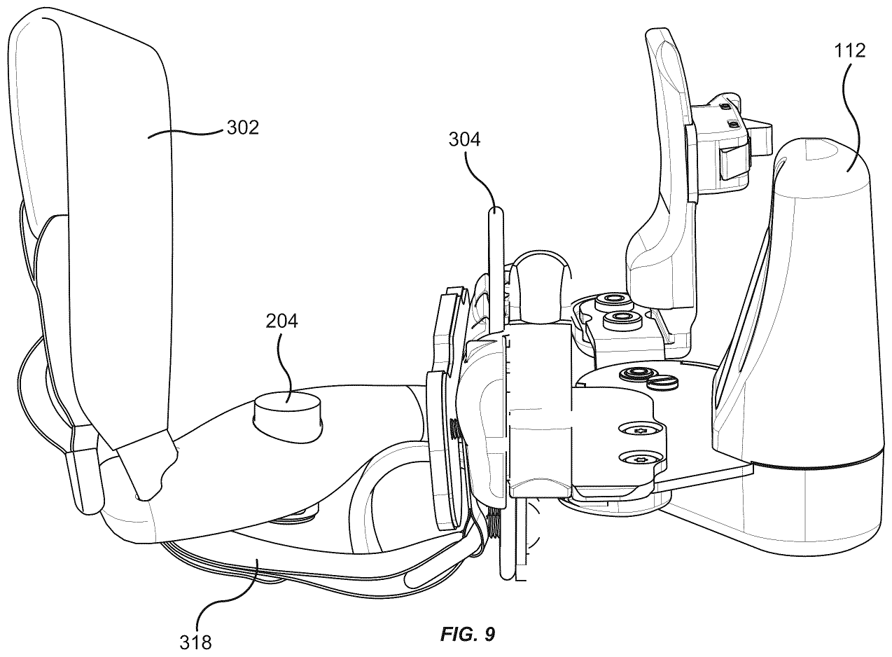

FIGS. 8 and 9 show the dorsal strapping arrangement 120 in an unstrapped configuration. The dorsal strap 302 passes between the dorsal and volar side of the hand shell 102. On the volar side, the thenar loop 318, formed from the volar segment of the dorsal strap 302 attaches to the volar mounting feature 320 disposed so as to position the thenar loop 318 across the base of a user's thumb when the user's hand is secured by the dorsal strapping arrangement 120.

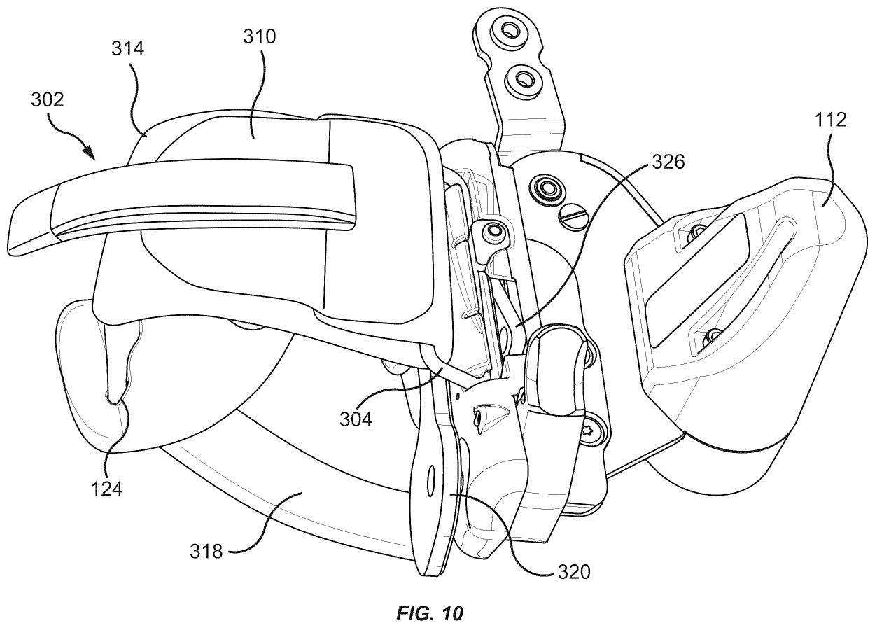

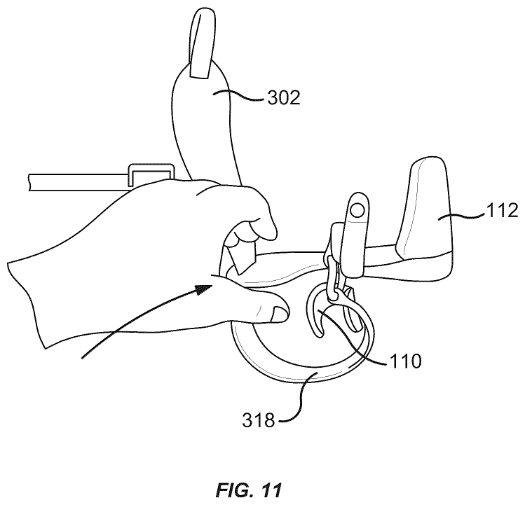

FIG. 10 shows the dorsal strapping arrangement 120 in a strapped configuration. In the strapped configuration the dorsal strap 302 passes through the dorsal ring 304, folds back on itself, and removably attaches to itself by the hooks on the dorsal alligator tab 310 and the loops of the dorsal strap loop material 314. The dorsal configuration allows for top-donning, according to which, as shown in FIG. 11, a user's hand is placed palm-side down on the palm region 106 of the hand shell 106, with the thumb inserted in the thumb shell 110 and the base of the thumb through the thenar loop 318. With the user's hand thus disposed, wrapping the dorsal strap 302 through the dorsal ring 304, tightening the dorsal strap 302 and attaching it back on itself secures the user's hand between the dorsal strap 302 and the palm region 106 of the hand shell 102, with the base of the thumb secured by the thenar loop 318.

FIG. 12 shows the thenar strapping arrangement 122 in an unstrapped configuration. The thenar strap 306 passes between the volar and the dorsal side of the hand shell 102. On the dorsal side, the dorsal loop 322, formed as the dorsal segment of the thenar strap 306 attaches to the dorsal mounting feature 326, disposed so as to position the dorsal loop 322 around the dorsal part of the user's hand when the user's hand is secured by the thenar strapping arrangement 122.

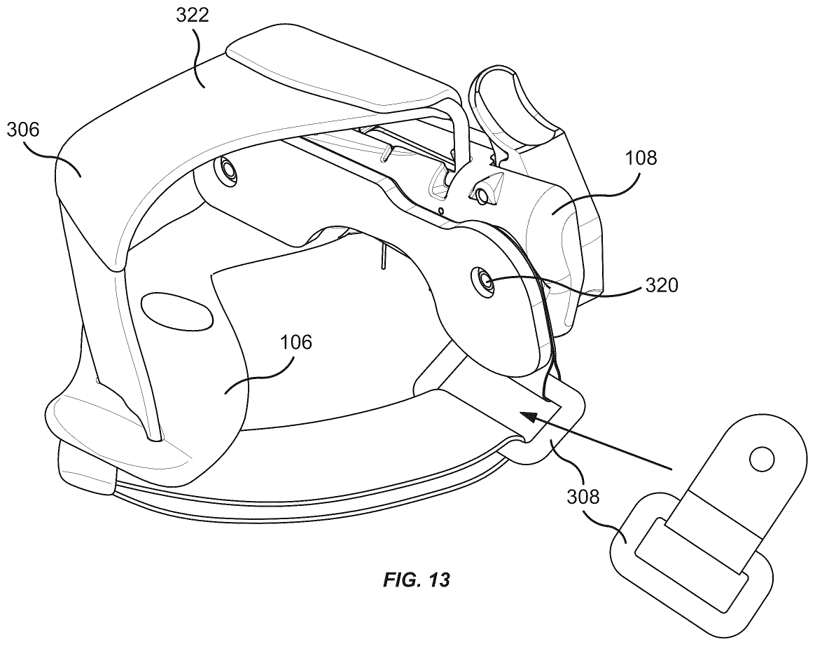

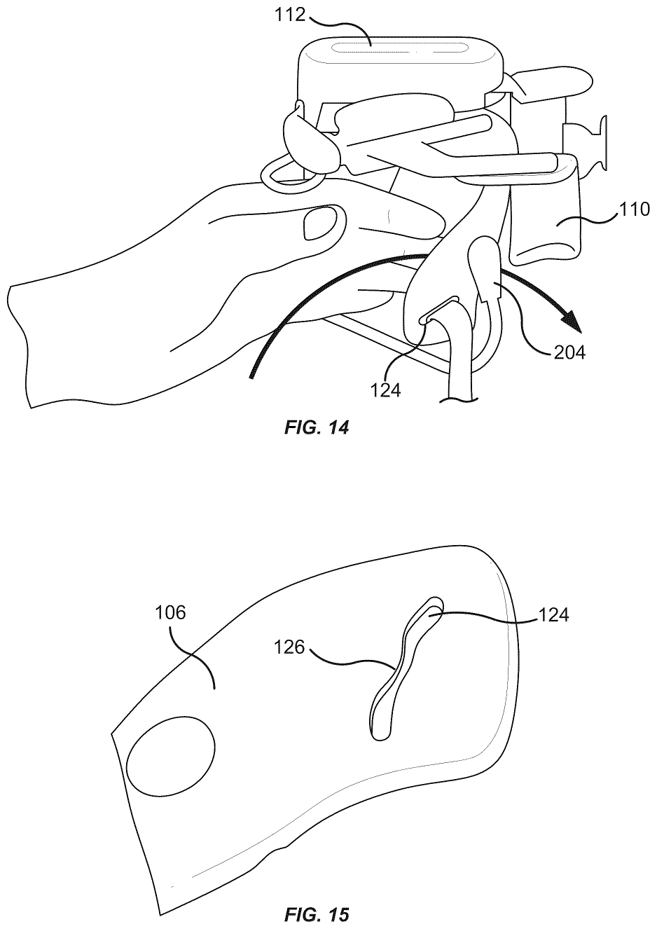

FIG. 13 shows the thenar strapping arrangement 122 in a strapped configuration. In the strapped configuration the thenar strap 306 passes through the volar ring 308, folds back on itself, and removably attaches to itself by the hooks on the thenar alligator tab 312 (FIG. 12) and the loops of the thenar strap loop material 316 (FIG. 12). The thenar configuration allows for slide-through donning, according to which, as shown in FIG. 14, a user's hand is slid between the dorsal loop 322 and the palm region 106 of the hand shell 102, with the palm of the hand on the palm region 106 of the hand shell 102, the dorsal loop 322 covering the dorsal part of the user's hand, and the thumb inserted in the thumb shell 110. With the user's hand thus disposed, wrapping the thenar strap 306 through the volar ring 308, tightening the thenar strap 306 and attaching it back on itself secures the base of the user's thumb with the thenar strap 306 and the user's hand between the dorsal loop 322 and the palm region 106 of the hand shell 102.

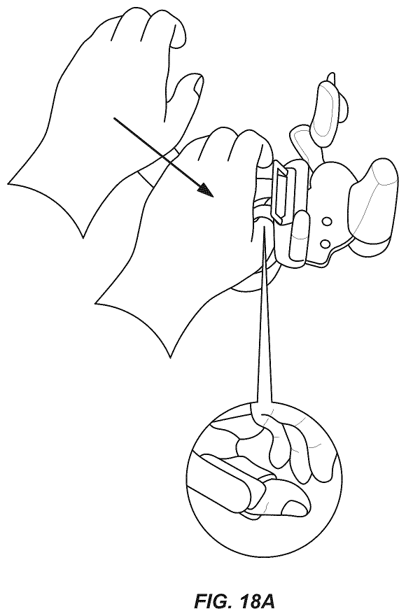

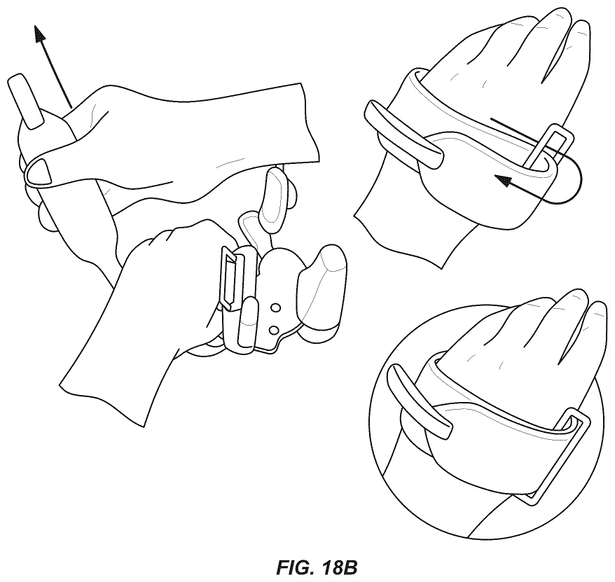

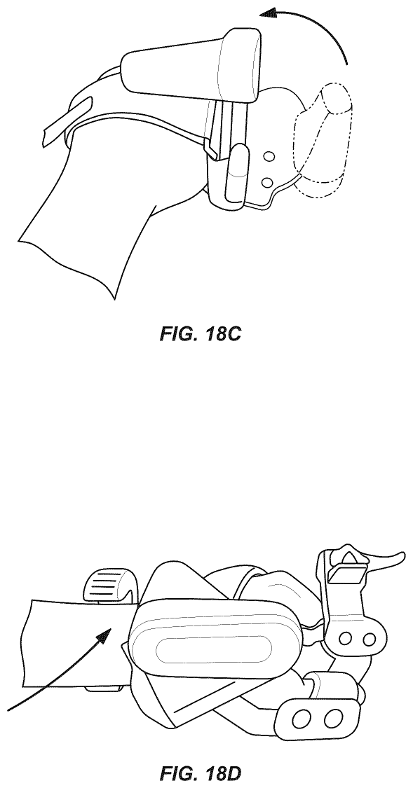

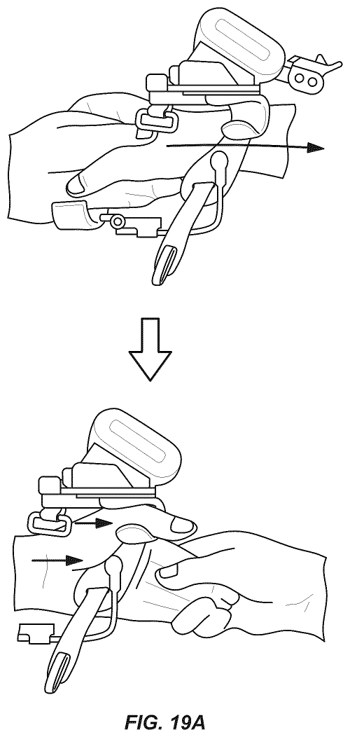

In the embodiment of FIG. 18, a dorsal strapping procedure may include the following steps.
(a) With the grasp actuator 112 unlatched and rotated to the radial position with respect to the hand shell 102, the user's hand is placed palm-down on the palm region 106 of the hand shell 102, with the index finger abutting the radial region 108 of the hand shell 102, and the user's thumb situated in the thumb shell 110 and engaged by the volar segment of the dorsal strap 302.
(b) The dorsal strap 302 is folded over the dorsal part of the hand, and threaded through the dorsal ring 304, and thence back on itself, where it is removably attached to itself by the hooks of the dorsal alligator tab 310 and the loops of the dorsal strap loop material 314.
(c) The grasp actuator 112 is rotated from the radial position to the dorsal position with respect to the hand shell 102, and latched in the closed configuration.
(d) The user's arm is rotated into place in the wrist shell 202 by swiveling the palm region 106 and strapped in hand about the rotatable palm swivel joint 204.

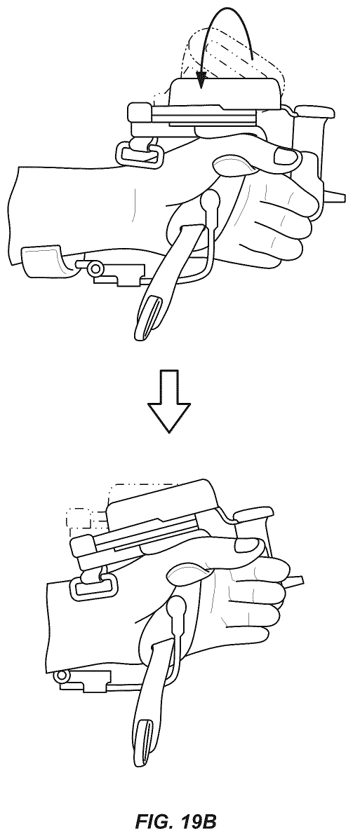

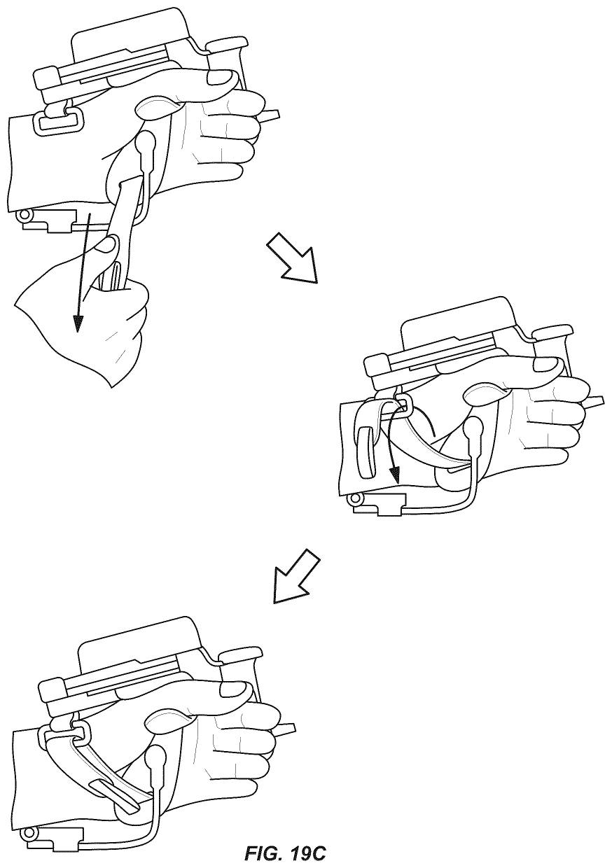

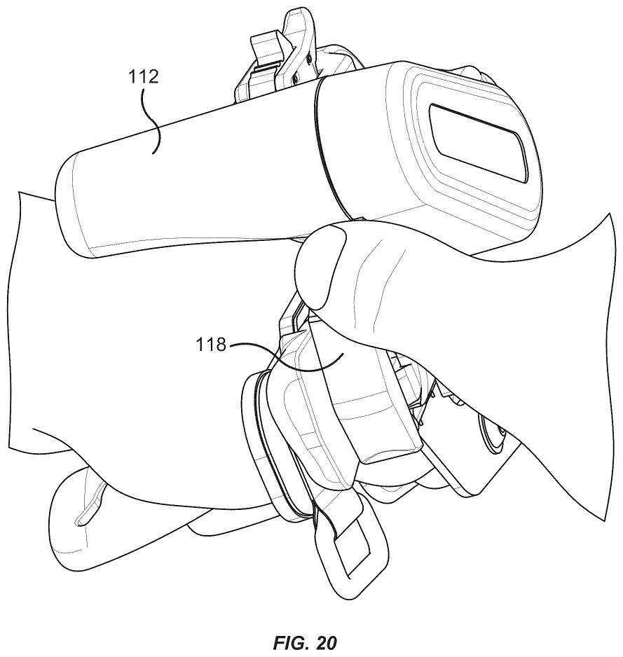

As shown in FIG. 19, a thenar strapping procedure may include the following steps:
(a) With the grasp actuator 112 unlatched and rotated to the open position with respect to the hand shell 102, the user's hand is slid between the dorsal segment of the thenar strap 306 and the palm region 106 of the hand shell 102, positioning the thumb in the thumb shell 110 and the user's arm in the wrist shell 202.
(b) The grasp actuator 112 is rotated from the radial position to the dorsal position with respect to the hand shell 102, and latched in place.
(c) The thenar strap 306 is folded over the base of the thumb, and threaded through the volar ring 308, and thence back on itself, where it is removably attached to itself by the hooks of the thenar alligator tab 312 and the loops of the thenar strap loop material 316.

The hand module 100 may be scalable to accommodate a wide range of anatomical dimensions, e.g. from a 10^{th} percentile sized female to a 90^{th} percentile sized male. The rotatable palm swivel joint 204 allows the user to angularly reposition the hand module 100 to suit the user's preferences for donning within a preset range of motion. The hand strapping system 104 provides accommodative containment of the user's hand in the radial-ulnar, distal-proximal, and dorsal-volar axes via use of a single tensioned strap that routes along the dorsal region of the hand from the radial side, wrapping around the ulnar side of the hand, and securing to the base of the thumb. The strapping system features a “checkpoint” approach which helps prevent a user from losing progress by maintaining the position of the strap post-tensioning via a frictional hold through the ulnar-side slot. The hand module 100 can be configured for either of two different approaches to donning by affixing one end of a strap to mounts on the hand shell 102 and attaching an alligator tab to the opposite end of the strap to allow for feeding and securement through the dorsal ring 304 in the dorsal strapping arrangement 120 or through the volar ring 308 in the thenar strapping arrangement 122. The dorsal strapping arrangement 120 uses the dorsal ring 304 for strap closure to allow the user to don the hand module 100 from above when the strap is opened, dropping the palm on top of the hand shell 102 with the base of the thumb engaged by the thenar loop 318 around the thenar eminence. The thenar strapping arrangement 122 uses the volar ring 308 for strap closure to allow the user to don the hand module 100 by feeding the hand through the dorsal loop 322. Both configurations proceed to secure the user's hand to the device when the free end of the strapping is tensioned, fed through the associated ring, and subsequently fastened via the hook alligator tab.

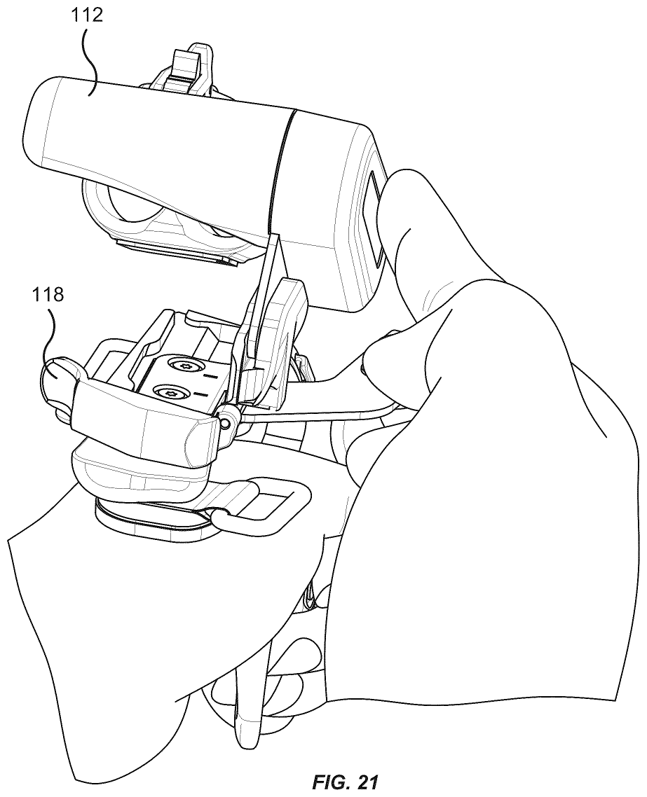

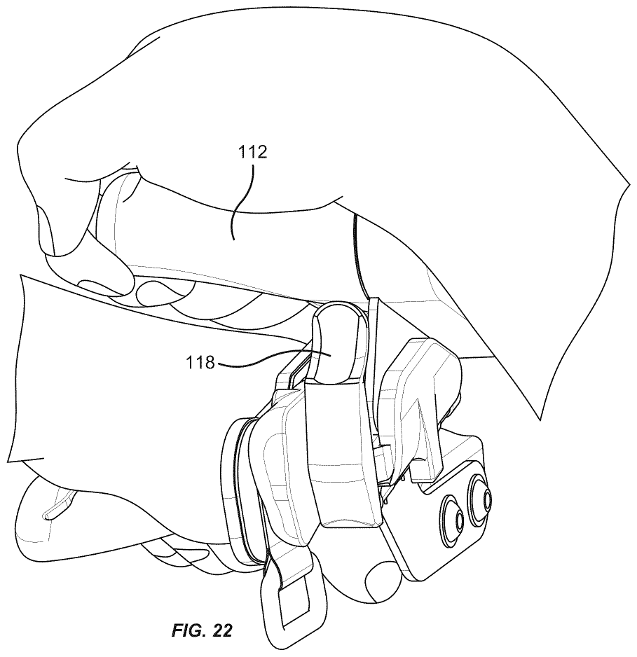

As illustrated in FIGS. 20 and 21 the grasp actuator 112 is equipped with an opening mechanism that allows the grasp actuator 112 to transition between the closed position shown in FIG. 20 and the open position shown in FIG. 21. The closed configuration is latched, and allows operation of the grasp actuator 112 to cause relative motion of the user's set of fingers and thumb. The open configuration facilitates donning by allowing the hand to be moved into place with the palm received by the palm region 106 of the hand shell 102, the set of fingers in the finger shell 114, and the thumb received by the thumb shell 110.

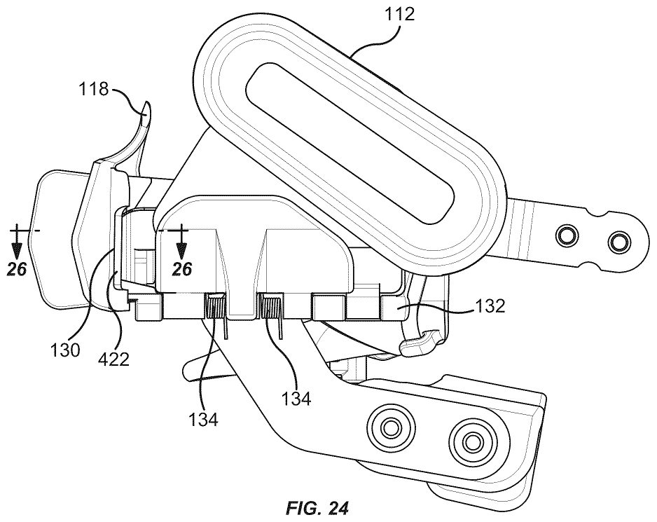

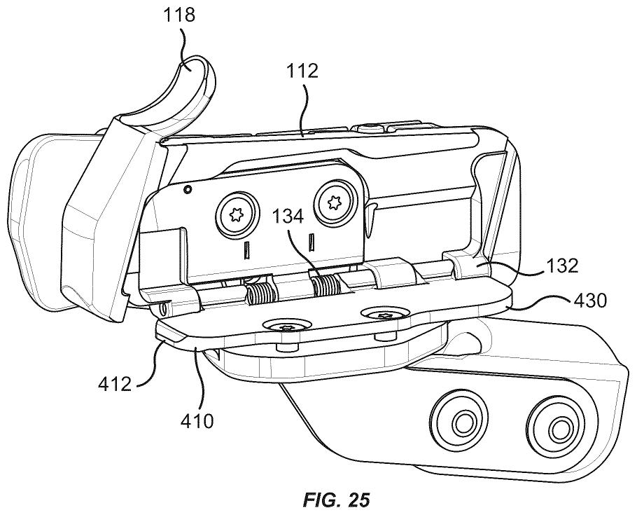

The transition between closed and open configurations can be effected by the user pressing a free thumb on the actuator lever 118, as shown in FIG. 20. Pressing the thumb on the actuator lever 118 in this manner causes the grasp actuator 112 to unlatch and thus allow the grasp actuator 112 to move to the open position as shown in FIG. 21 due to torsional force provided by at least one spring connected to the grasp actuator 112 and the radial region 108, e.g., as discussed below with respect to FIGS. 24 and 25.

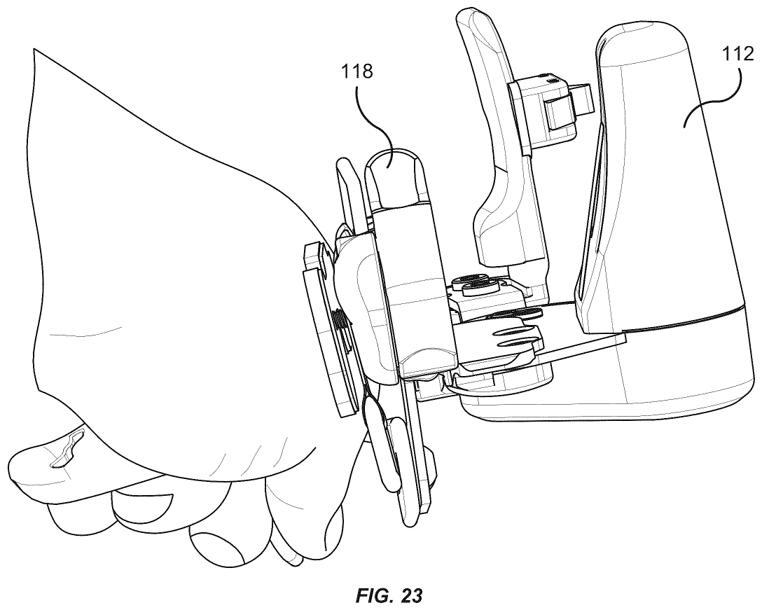

Taking into account the likelihood that a user might forget to press the actuator lever 118 in preparation for donning and instead attempt to pull the grasp actuator 112 open, a latching mechanism of the grasp actuator 112 is further configured to allow the grasp actuator 112 to be pulled into the open position without damaging the grasp actuator 112 or the latching mechanism. Shown in FIG. 22, the user's free hand is positioned on the grasp actuator 112 and begins pulling on it. When the force applied by the user's free hand exceeds a threshold value (e.g., 13 Newtons), the grasp actuator 112 moves to the open position suitable for donning. The latching mechanism also helps prevent damage to the grasp actuator 112 or the latching mechanism if the grasp actuator 112 is closed without the actuator lever 118 being pressed. The force to close the grasp actuator 112 without pressing the actuator lever 118 may be less than the force to open the grasp actuator 112 without pressing the actuator lever 118.

Referring also to FIGS. 24 and 25, the grasp actuator 112 may be equipped with a cam latch 130 and with a hinge 132 having torsional springs 134. In the closed, latched configuration of the grasp actuator 112 shown in FIG. 24, the torsional springs 134 are taut, but are held in place by the cam latch 130. Pressing the actuator lever 118 releases the cam latch 130, so that the tension on the torsional springs 134 is released, causing the grasp actuator 112 to rotate about the hinge 132 to the open configuration shown in FIG. 25. The cam latch 130 can also be released by pulling the grasp actuator 112 towards the open position. When the grasp actuator 112 is pulled in this manner, with a force exceeding a threshold value (e.g. 13 Newtons), surface features on the cam latch 130 push the actuator lever 118 open, releasing the tension on the torsional springs 134, causing the grasp actuator 112 to continue to rotate about the hinge 132 to the open position shown in FIG. 25. In this manner, the grasp actuator 112 can be opened either by pressing the actuator lever 118 or by pulling the grasp actuator 112 towards the open position without first pressing the actuator lever 118. In the latter case, the smooth operation of the cam latch 130 helps ensure that the forced opening by pulling on the grasp actuator 112 does little or no damage to the grasp actuator 112 or the latching mechanism.

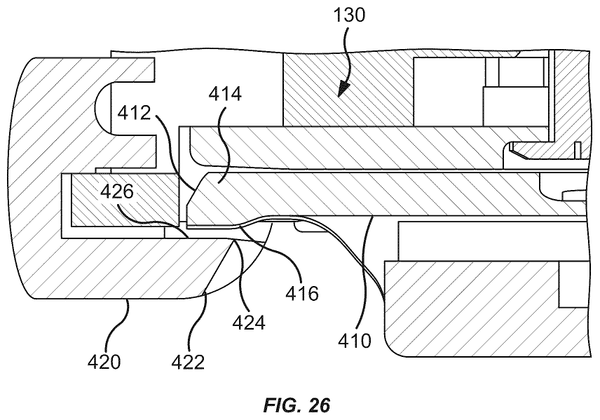

Referring also to FIG. 26, which is a cross-sectional view taken along line 26-26 shown in FIG. 24, the cam latch 130 includes a flexible arm 410 and a catch plate 420. As shown, the cam latch 130 is in a closed position, with the grasp actuator 112 in a closed position. The flexible arm 410 is an end portion of a plate 430 shown in FIG. 25. The catch plate 420 is a portion of the actuator lever 118 that is configured to interact with the flexible arm 410 to resist closing and opening of the grasp actuator 112 without pressing the actuator lever 118, resisting opening more than resisting closing of the grasp actuator 112. The flexible arm 410 includes a cam surface 412 (also shown in FIG. 25) that is angled relative to a length of the flexible arm 410 and is angled similarly to a cam surface 422 (also shown in FIG. 24) of the catch plate 420. The cam surfaces 412, 422 will engage while the grasp actuator 112 is being closed, causing an end 414 of the flexible arm 410 to bend, and the surface 412 to slide along the surface 422 until the end 416 passes over a tip 424 of the catch plate 420. With the grasp actuator 112 in the closed position and forced toward the open position, a cam surface 416 that is parallel with a length of the flexible arm 410 will engage with a cam surface 426 of the catch plate 420, causing the flexible arm 410 to bend until the end 414 passes over the tip 424. Due to the cam surfaces 412, 422 being angled relative to the direction of motion of the end 414 while the grasp actuator 112 is being closed, and the cam surfaces 416, 426 being substantially perpendicular to the direction of motion of the end 414 while the grasp actuator 112 is being opened, the cam latch 130 resists forced opening of the grasp actuator 112 much more than the cam latch 130 resists forced closing of the grasp actuator.

The embodiments of the invention described above are intended to be merely exemplary; numerous variations and modifications will be apparent to those skilled in the art. All such variations and modifications are intended to be within the scope of the present invention as defined in any appended claims.

As used herein, the singular forms “a,” “an,” and “the” include the plural forms as well, unless the context clearly indicates otherwise.

The terms “comprises,” “comprising,” “includes,” and “including,” as used herein, specify the presence of stated features but do not preclude the presence of other features.

What is claimed is:

1. An orthotic device having a hand module configured for strapping a hand of a user, and for effectuating relative movement of a thumb of the hand and a set of fingers of the hand, the hand module comprising:
a hand shell, configured to receive a palm of the hand;
a finger shell, coupled to the hand shell, and configured to receive the set of fingers;
a thumb shell, coupled to the hand shell, and configured to receive the thumb, wherein the finger shell and the thumb shell are configured to support relative motion of the thumb and the set of fingers;
a grasp actuator coupled to the finger shell and the thumb shell, and configured to cause relative motion of the set of fingers and the thumb;
a hand strapping system for securing a dorsal side of the user's hand and a base of the user's thumb, the hand strapping system including mounted hardware to support (i) a dorsal strapping arrangement for top-down donning and (ii) a thenar strapping arrangement for slide-through donning.

2. The orthotic device of claim 1, wherein:
the dorsal strapping arrangement includes:
a dorsal ring, removably attached on a radial region of the hand shell;
a thenar loop, configured to engage the base of the user's thumb with the user's thumb received in the thumb shell; and
a dorsal strap configured to engage against the dorsal side of the hand and to be looped through the dorsal ring, and thence back to itself, where the dorsal strap can be removably attached, so as to secure the hand between the dorsal strap on the dorsal side of the hand and the hand shell on the palm of the hand; and
the thenar strapping arrangement includes:
a volar ring, removably attached on the radial region of the hand shell;
a dorsal loop, configured to engage against the dorsal side of the user's hand with the palm received on the hand shell; and
a thenar strap configured to wrap around the base of the thumb and to be looped through the volar ring, and thence back to itself, where the thenar strap can be removably attached, so as to secure the thumb in the thumb shell.

3. The orthotic device of claim 2,
wherein the hand shell has an ulnar region, configured such that with the hand of the user secured in the hand module, the ulnar region is disposed adjacent to a little finger of the hand, the ulnar region having an ulnar slot configured to allow passage of a strap inserted therein,
wherein in the dorsal strapping arrangement, the dorsal strap is configured to pass through the ulnar slot, in a dorsal to volar direction, and to be affixed to the radial region of the hand shell, thereby forming the thenar loop, and
wherein in the thenar strapping arrangement, the thenar strap is configured to pass through the ulnar slot, in a volar to dorsal direction, and to be affixed to the radial region of the hand shell, thereby forming the dorsal loop.

4. The orthotic device of claim 3, the ulnar slot having a perimeter with features configured to provide a frictional hold on the dorsal strap with the dorsal strapping arrangement and on the thenar strap with the thenar strapping arrangement.

5. The orthotic device of claim 4, wherein the features configured to provide the frictional hold are teeth.

6. The orthotic device of claim 2, the orthotic device having a dorsal mounting feature, the dorsal ring being attached to the dorsal mounting feature in the dorsal strapping arrangement.

7. The orthotic device of claim 6, wherein for the thenar strapping arrangement the dorsal loop is attached to the dorsal mounting feature.

8. The orthotic device of claim 2, the orthotic device having a volar mounting feature, the volar ring being attached to the volar mounting feature in the thenar strapping arrangement.

9. The orthotic device of claim 8, wherein for the dorsal strapping arrangement the thenar loop is attached to the volar mounting feature.

10. The orthotic device of claim 7 wherein the dorsal mounting feature has a dorsal ring tightening mechanism which allows a frictional force on the dorsal ring to be adjusted, allowing the dorsal ring to be held in an open position as the dorsal strap is passed through it.

11. The orthotic device of claim 2, wherein the dorsal strap and the thenar strap are each configured with a hook-and-loop mechanism to allow the dorsal strap to removably attach to itself, and the thenar strap to removably attach to itself.

12. The orthotic device of claim 1, further comprising:
a wrist shell configured to secure a wrist of the user with the hand secured in the hand shell; and
a hand to wrist coupler connecting the wrist shell to the hand shell by a palm swivel joint on the hand shell, the palm swivel joint configured to allow swiveling of the hand about a dorsal to volar axis centered at the palm swivel joint.

13. An orthotic device having a hand module configured for strapping a hand of a user, and for effectuating relative movement of a thumb of the hand and a set of fingers of the hand, the hand module comprising:
a hand shell configured to receive a palm of the hand;
a finger shell, coupled to the hand shell, and configured to receive the set of fingers;
a thumb shell, coupled to the hand shell, and configured to receive the thumb;
wherein the finger shell and the thumb shell are configured to support relative motion of the thumb and the set of fingers;
a hand strapping system configured to secure the hand in the hand module;
a grasp actuator having a pivotal attachment to a radial region of the hand shell, the pivotal attachment being configured to allow the grasp actuator to pivot about a hinge from a closed configuration to an open configuration, and to hold the grasp actuator in place in the closed configuration and in the open configuration;
the open configuration allowing the hand to be moved into place with the palm received by the hand shell, the set of fingers received by the finger shell, and the thumb received by the thumb shell, and
the closed configuration allowing operation of the grasp actuator to cause relative motion of the set of fingers and the thumb;
an actuator lever mechanically coupled to the grasp actuator; and
a self-opening mechanism configured to move the grasp actuator from the closed configuration to the open configuration in response to a trigger action by the user.

14. The orthotic device of claim 13 wherein the trigger action is selected from the group consisting of pressing the actuator lever and applying a force in an opening direction to the grasp actuator.

15. The orthotic device of claim 14, wherein the applied force is at least 13 Newtons.

16. The orthotic device of claim 13, wherein the self-opening mechanism is equipped with torsional springs about the hinge, the torsional springs being taut in the closed configuration of the grasp actuator, wherein application of the action releases tension on the torsional springs, causing the grasp actuator to transition from the closed configuration to the open configuration.

17. The orthotic device of claim 13, wherein the self-opening mechanism is effectuated by a cam latch.

18. A method of securing a hand of a user in the hand module of an orthotic device according to claim 2, the method comprising:
pursuant to choosing the dorsal strapping arrangement:
placing the palm of the hand on a palm region of the hand shell;
positioning the thumb through the thenar loop into the thumb shell;
passing the dorsal strap over a dorsal region of the hand;
inserting the dorsal strap in a radial direction through the dorsal ring;
passing the dorsal strap through the dorsal ring;
folding the dorsal strap back towards the little finger;
pulling the dorsal strap towards the little finger; and
attaching the dorsal strap to itself, thereby securing the hand in the hand module.

19. A method of securing a hand of a user in the hand module of an orthotic device according to claim 2, the method comprising:
pursuant to choosing the thenar strapping arrangement:
sliding the hand of the user between the dorsal loop and a palm region of the hand shell;
positioning the thumb of the user in the thumb shell;
passing the thenar strap around the base of the thumb;
inserting the thenar strap in a radial direction through the volar ring;
passing the thenar strap through the volar ring;
folding the thenar strap back towards the little finger;
pulling the thenar strap towards the little finger; and
attaching the thenar strap to itself, thereby securing the hand in the hand module.
